(19) United States
(12) Patent Application Publication
(10) Pub. No.: US 2025/0228565 A1
(43) Pub. Date: Jul. 17, 2025

(54) CATHETER SYSTEM AND METHOD FOR CLOSURE OF AT LEAST ONE PARAVALVULAR LEAKAGE

(71) Applicant: BIOTRONIK AG, Buelach (CH)

(72) Inventors: Georg Nollert, Strasslach (DE); Tobias Kissling, Bern (CH)

(21) Appl. No.: 18/881,838

(22) PCT Filed: Jul. 7, 2023
(86) PCT No.: PCT/EP2023/068848

§ 371 Date: Jan. 7, 2025

(30) Foreign Application Priority Data
Jul. 15, 2022 (EP) 22185072.0

(51) Int. Cl. A61B 17/12; A61B 17/00; A61B 90/00; A61F 2/24

(52) U.S. Cl. CPC A61B 17/12122; A61B 17/00234; A61B 17/1214; A61B 90/06; A61B 2017/00238; A61B 2017/00305; A61B 2017/00367; A61B 2017/1205; A61B 2090/064; A61F 2/24

(57) ABSTRACT

A catheter system for a interventional procedure includes a bendable and/or steerable extension arm and a guiding sheath. The extension arm is movable along the sheath or fixed at the distal end so that it projects in an operation position from the distal end. The extension arm has an inner guidewire lumen. At least one alignment component having a collapsed state and being configured to expand into an expanded state is advanceable to or attached to the guiding sheath at a pre-defined position within the distal section but proximally from its distal end. The alignment component includes a plurality of struts distributed on the circumference. Each strut of the struts runs at least sectionwise into radial direction and/or is inclined with regard to the longitudinal axis in the expanded to produce a force directed radially outwardly and to support the alignment component on the patient's heart, vasculature and/or implant.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/068848, which was filed Jul. 7, 2023, which application claimed priority from EP Application 22185072.0, which was filed Jul. 15, 2022.

### FIELD OF THE INVENTION

A field of the invention is systems for an interventional procedure, within a patient's heart, vasculature and/or an implant fixed at the patient's heart or vasculature.

### BACKGROUND

Cardiovascular disease refers to a class of diseases that involve the heart or blood vessels (arteries and veins) and include stenotic heart valves, wherein the valve does not open sufficiently to allow adequate forward flow of blood through the valve, and/or incompetent heart valves, wherein the valve does not close completely. An effective therapy for these conditions is valve replacement which could be realized by an interventional or endovascular procedure, such as percutaneous valve replacement. Paravalvular leakage (PVL) is a complication associated with the implantation of a prosthetic heart valve that may also occur other implants containing a valve. PVL or paraprosthetic leakage refers to blood flowing through a channel (a leak) between the structure of the implant containing the valve and the tissue of the heart or vasculature in which the implant with the valve is fixed as a result of a lack of appropriate sealing. The PVL may cause blood backflow or clotting of blood in the implant region and thereby entail the risk of heart failure, hemolytic anemia and infectious endocarditis. It is believed that severe paravalvular aortic regurgitation is a key reason for prosthetic valve dysfunction. Significant PVLs can be treated either surgically or using the transcatheter deployment of occluding members. The latter is particularly offered to patients with isolated PVL or to those with a very high operative risk.

Document US 2016/0367120 A1 discloses a catheter with a distal cardioscope that is used for the detection and repair of leaks around a replacement valve in a heart. The catheter including the distal cardioscope is inserted into the heart through an introducer sheath placed through the apex of the heart and navigated through the left ventricle and to the periphery of the aortic annulus. The optical window of the cardioscope enables visualization of the positioning of the distal tip of the catheter relative to the valve and possibly visualization of the leak itself. For each PVL, a wire is passed through the leak and an occluder device is deployed along the wire to repair the PVL. This method seems to be hazardous to patients with a higher surgical risk due to comorbidity and requires elaborate imaging technology.

Document US 2019/0374326 A1 discloses an expandable material selected from the group consisting of hydrogels, sponges and foams and a membrane adjacent to and containing the expandable material, wherein the expandable material is activated by exposure to a fluid or a foaming agent. However, with regard to this method, a cost-effective way is desired to identify the PVLs which need to be closed.

### SUMMARY OF THE INVENTION

Preferred embodiments provide a catheter system that provides for the closure of at least one paravalvular leakage and that allows for reliable and efficient placement of an occluding member optimized for sealing PVLs, which, for example, does not need elaborate imaging technology.A preferred catheter system for a interventional procedure, within a patient's heart, vasculature and/or an implant fixed at the patient's heart or vasculature, includes a bendable and/or steerable extension arm and a guiding sheath with a distal section. The extension arm is fixed at the distal end of the distal section of the guiding sheath or is configured to be moved along the guiding sheath so that it projects in an operation position from the distal end of the distal section. The extension arm has an inner lumen through which a guidewire can be maneuvered and defines a longitudinal axis. At least one alignment component has a collapsed state and is configured to expand into an expanded state. The alignment component is attached to the guiding sheath at a pre-defined position within the distal section but proximally from its distal end or the alignment component is configured to be advanced along the guiding sheath to this pre-defined position within the distal section. At least one guidewire is within the inner lumen of the extension arm and an occluding member is in contact with a distal guidewire section and/or a distal guidewire end of the at least one guidewire. The alignment component may include a plurality of struts distributed on the circumference. Each strut of the plurality of struts preferablyt runs at least sectionwise into radial direction and/or is inclined with regard to the longitudinal axis in the expanded state thereby producing a force directed radially outwardly and supporting the alignment component on the patient's heart, vasculature and/or implant. Additionally or alternatively, the alignment component may include an inflatable structure, such as a balloon, allowing for perfusion of blood. The alignment component can be a toroidal balloon.

### BRIEF DESCRIPTION OF THE DRAWINGS

The various features and advantages of the present invention can be more readily under-stood with reference to the following detailed description and the embodiments shown in the drawings. Herein schematically and exemplarily,

FIG. 1 depicts a distal end of a first embodiment of a catheter system in a side view;

FIG. 2 shows a second embodiment of the catheter system in a side view with the alignment component in an expanded state;

FIG. 3 shows the embodiment of FIG. 2 with the alignment component in a collapsed state in a side view;

FIG. 4 visualizes the alignment component of the embodiment of FIG. 1 in a top view;

FIG. 5 shows a second embodiment of an alignment component in a top view,

FIG. 6 depicts a third embodiment of an alignment component in a side view;

FIG. 7 shows a distal section of a third embodiment of a catheter system in a side view, wherein the alignment component is in an expanded state;

FIG. 8 depicts the embodiment of FIG. 7 in a collapsed state in a side view;

FIG. 9 shows a distal end of a fourth embodiment of a catheter system in a side view, wherein the alignment component is in an expanded state;

FIG. 10 shows a distal end of a fifth embodiment of a catheter system in a side view, wherein the alignment component is in an expanded state;

FIG. 11 visualizes the embodiment of the catheter system of FIG. 1 and a stent of a prosthetic aortic valve prosthesis in a side view;

FIGS. 12-14 show three steps of an embodiment of a method for closure of at least one PVL at a transition area between the heart and a prosthetic aortic valve in a side view; and

FIGS. 15-18 show four steps of another embodiment of a method for closure of at least one paravalvular leakage at a prosthetic aortic valve in a top view.

### DETAILED DESCRIPTION OF THE PREFERRED EMBODIMENTS

Preferred catheter systems can be used to look for and identify the position of a leak within the heart, the vasculature and/or within the transition area between the heart or the vasculature and the implant in a precise and efficient way and to easily and reliably position an occluding member within the leak at the identified position thereby closing the leak.

A preferred catheter system includes a guiding sheath, for example a tubular member, and an extension arm fixed at a distal end of the distal section of the guiding sheath or extends therefrom in an operation position. The extension arm may also be formed as a tubular member in order to provide an inner lumen through which the guidewire can be maneuvered. The guiding sheath further defines a longitudinal axis which may correspond to the longitudinal axis of the extension arm when the extension arm is straight. The catheter system further may include an outer sheath, for example a tubular member, with an inner lumen in which the guiding sheath and the collapsed alignment component is guided to the targeted area within the heart or vasculature of the patient. The guiding sheath can be configured to pass through the implant fixed within the heart or vasculature.

The extension arm can be bendable and/or steerable and/or rotatable, for example at a distal section close to and/or at the distal tip in order to provide a flexible extension arm. The distal tip may form an exit port. A steerable extension arm may have the advantage that the distance between the longitudinal axis of guiding sheath and the distal tip of the extension arm can be adjusted thereby allowing for individual adjustment to the diameter of the implant, the vasculature or the heart of the patient. The extension arm can be operated from the proximal end of the catheter system, for example by a steering wire, and thereby provides flexibility to look for and identify the position of a leak within the heart, the vasculature and/or within the transition area between the heart or the vasculature and the implant. The extension arm may have a preset shape, e.g. a J-shape or a U-shape, and can be straightened during advancement of the system through the vasculature to the targeted area. Straightening may occur through a stiff guidewire from the inside or through an outer sheath from the outside. When the straightening force is removed by relative axial displacement between the extension arm and the straightening structure, the extension arm will adopt the preset J- or U-shape. The steerable section may, e.g., be formed from a lasercut hypotube and steering wires can be used to control bending and direction of the extension arm. Any suitable type of steerable catheter constructions can be adopted at the bendable and/or steerable distal section of the extension arm. In one embodiment, the guiding sheath is bendable by at least 120° so that by the guiding sheath leaks can be identified which are located backwards (i.e. proximally) from the distal tip of the extension arm. In one embodiment, the extension arm can be manually or automatically rotatable around its longitudinal axis relative to the guiding sheath in order to provide further flexibility and maneuverability in order to identify leaks and close them. In one embodiment, the extension arm is configured such that it is rotatable during looking for a position of a leak within the heart, the vasculature and/or within the transition area between the heart or the vasculature and the implant. If the extension arm is attached to the distal end of the guiding sheath thereby forming a distal part of the guiding sheath, a rotatable connection between the guiding sheath and the extension arm can be required. A motor for actuating the rotation can be provided at the rotatable connection. If the extension arm is attached to the distal end of the guiding sheath, this structure can be rotated relative to the guiding sheath, e.g. by a control structure (handle, robot, etc.) at the proximal part of the system. The tubular extension arm may provide an exit port at its distal tip.

The at least one alignment component has a collapsed state and an expanded state to which it can be expanded from the collapsed state. Further, the at least one alignment component is attached to the guiding sheath at a pre-defined position within the distal section but proximally from its distal end or the alignment component is configured to be advanced along the guiding sheath to this pre-defined position within the distal section. Thereby, the alignment component has a pre-defined distance (with regard to the longitudinal direction) from the extension arm and does not interfere with the function of the implant at which the PVL can be identified but defines the position of the extension arm within the heart, vasculature and/or implant as indicated below. The alignment component is realized by a member consisting of struts, wherein at least a part of the struts run at least sectionwise into radial direction and/or inclined with regard to the longitudinal axis. The alignment component is neither inflatable nor a balloon. Accordingly, the alignment component may include a plurality of struts distributed on the circumference, wherein in the expanded state each strut of the plurality of struts runs at least sectionwise into radial direction and/or inclined with regard to the longitudinal axis thereby producing a force directed radially outwardly and supporting the alignment component on the patient's heart, vasculature and/or implant. In one embodiment, the length of the struts can be adapted to the anatomical situation of the patient and/or the dimensions of the implant.

The catheter system may include one alignment component or two or more than two alignment components, wherein, two or more alignment components are provided, they are arranged side by side along the longitudinal axis of the guiding sheath. The at least one alignment component functions as a stabilizing and centering aid in the expanded state for the catheter system, in particular its distal end, which is achieved by the radial force provided by the plurality of struts. Accordingly, in one embodiment, the alignment component is configured to keep the distal end of the guiding sheath in a pre-defined position, for example an approximately central position or a position having a pre-set distance from the central position, with regard to the surrounding heart, vasculature or implant. Thereby, the position of the extension arm which is fixed to the distal end of the guiding sheath or projects from the distal end of the guiding sheath in an operation position is stabilized and centered (in a central position or at a pre-set distance from the central position), as well. Therein, the operation position is the position of the extension arm in which it is configured to look for and identify the position of a leak within the heart, the vasculature and/or within the transition area between the heart or the vasculature and the implant and to position the occluding member within the leak at the identified position thereby closing the leak. For example, the alignment component can be positioned within the inflow or the outflow section of one heart valve prosthesis such that the alignment component is located with a distance from the artificial heart valve in order not to interfere with the functioning of the heart valve, in particular its leaflets.

Different embodiments refer to different constructions of the at least one alignment component. In one embodiment, the alignment component includes in the expanded state at least two spoke-like struts which extend radially from the guiding sheath and at least a section of a torus, circle, ellipse (in one embodiment a full torus, circle, ellipse) or the like connecting and being attached to the outer tips (tips pointing away from the longitudinal axis) of the at least two struts.

In another embodiment, the alignment component includes in the expanded state at least two struts which extend inclined outwards from the inner wall of the guiding sheath and at least a section of a torus, circle, ellipse (in one embodiment a full torus, circle, ellipse) connecting and being attached to the outer tips (tips pointing away from the longitudinal axis) of the at least two struts.

In another embodiment, the alignment component includes one distal sleeve and one proximal sleeve, both attached to the guiding sheath and at least two struts each extending between the distal sleeve and the proximal sleeve and having a curved form with one or a plurality of curves (e.g. forming a helix) extending outwardly from the longitudinal axis in the expanded state such that they together realize a bulbous or cylindrical outer form. In the collapsed state, the at least two struts run parallel to the longitudinal axis. In one embodiment of the last embodiment mentioned above two struts of the at least two struts can be fixed to a connecting member at the distal end of the struts, wherein such connecting member is configured such (with regard to the struts) that they provide a radial force to the heart, vasculature or implant, as well. For example, as indicated above, the at least one alignment component can be positioned within the inflow section, the outflow section of one heart valve prosthesis and/or in the ascending aorta. In one embodiment, the alignment component has an outer diameter that is in a range between 20 mm and 50 mm in the expanded state in order to be supported by the patient's heart, vasculature and/or implant, for example, by a heart valve chamber, the patient's main vasculature such as the aorta or a heart valve prosthesis.

In one embodiment, the catheter system further includes at least one guidewire within the inner lumen of the extension arm. In one embodiment, a floater is attached at the distal end of the guidewire. The floater can be used for identifying the position of a leak within the heart, the vasculature and/or within the transition area between the heart or the vasculature and the implant as described below in detail. In one embodiment the floater can be realized as a balloon which can be inflated e.g. with a solution including radiopaque contrast agent. Alternatively, an umbrella type floater can be used.

The occluding member can be used to close the identified leak within the heart, the vasculature and/or the implant or at the transition area between the heart or vasculature and the implant. The occluding member can be advanced to the targeted area using an occluding catheter and an occlude guidewire, wherein the occluding catheter is moved over the occluder guidewire. Any type of occluding member is suitable to close an identified lead. In one embodiment, a coil-type occluding member can be used (i.e. an occluding member including a coil). The coil may have a contracted or an expanded configuration. The coil can e.g. adopt a (bilobal) dumbbell-shape in the expanded configuration. The coil may expand and/or fix a sealing material at a treatment site. Alternatively, the coil may lead to sealing by thrombus formation on the coil. In another embodiment, a stent-based occluding member can be used. For example, a self-expanding or balloon-expandable stent can be used to place and fix a sealing material within the identified leak. A self-expanding frame can be advantageous and allow for better adaptation to the size and shape of an identified leak.

In one embodiment, the system can be pre-assembled including an occluding member delivery catheter pre-assembled in the guiding sheath and acting as a space-holder/dilator during introduction of the system into the vasculature. This provides several advantages: it facilitates the procedure since less handling steps are required, it improves safety of the procedure since less components are directly exposed to the cath-lab environment which reduces the risk of contamination with particles and/or infectious agents and it reduces the number of required parts thereby reducing manufacturing costs, storage space and environmental impact. In one embodiment, a variety of occluding members of different type, size and/or shape can be chosen depending on the size and shape of a specific identified leak. A dilator can be required for initial placement of the guiding sheath.

In one embodiment, an additional occluding member delivery catheter can be provided separately. After the first occluding member has been placed, the guiding sheath (and, if applicable, the guidewire) is left in place and only the used empty occluding member delivery catheter is removed. Then a (new) second occluding member delivery catheter is introduced through the same guiding sheath catheter and the closure procedure is performed at a second identified leak. Further leaks can be sealed accordingly.

Regarding the material of the components of the system, the struts of the alignment component may include or consist of shape memory material, e.g. Nitinol. The guiding sheath, the extension arm, the floater and the occluding member can be made of a biocompatible material (suitable for catheters). The biocompatible material can be a suitable polymer, metal and/or metal alloy.

The above object is further solved by a method for the closure of a paravalvular leakage at a transition area between the natural heart valve structure and a heart valve prosthesis using the catheter system described above

Such a method for the closure of at least one paravalvular leakage within a targeted area of an heart, a vasculature and/or a transition area between the heart or the vasculature and an implant fixed at the patient's heart or vasculature, preferably using the catheter system as described above, may have the following steps:
Advancing the distal section of the guiding sheath to the targeted area, wherein the alignment component is in the collapsed state,
Expanding the alignment component at a pre-defined position within the distal section of the guiding sheath and with regard to the implant and/or the heart or vasculature such that it is supported at the implant, heart and/or vasculature,
Using the bent and/or steered extension arm fixed at or projecting from the distal end of the distal section of the guiding sheath to look for and identify the position of a leak within the heart, the vasculature and/or within the transition area between the heart or the vasculature and the implant,
Advancing the occluding member through the extension arm to the leak at the identified position using, for example, a guidewire.

The position of the leak can be identified using the floater attached to a distal end of a guidewire and monitoring the tensile force acting on the guidewire and/or the extension arm.

The extension arm can be rotated during looking for a position of a leak within the heart, the vasculature and/or within the transition area between the heart or the vasculature and the implant.

The occluding member can be positioned within the leak at the identified position.

The above method steps and the elements used by the method are partly described with regard to the catheter system. It is therefore referred to the above explanation.

In one embodiment, prior advancing the guiding sheath to the targeted area, a guidewire, for example having an atraumatic tip, is advanced to the targeted area. The catheter system is then advanced to the targeted area over the guidewire. In one embodiment, the guidewire lumen is separate from the guiding sheath lumen. In another embodiment, the guidewire lumen is arranged in the center of the catheter system within the guiding sheath and the guidewire used for placement and advancement of the catheter system at the targeted area may further be used as guidewire for placement of the occluding member after deployment of the extension arm.

In one embodiment, the position of the leak is identified using the floater attached to a distal end of a guidewire and monitoring the tensile force acting on the guidewire or the force deforming the extension arm. This method is based on the observation that at the location of an PVL a strong flow is present which exerts a force on the floater attached to the guidewire if it is placed in this flow (also referred to as PVL jet). The force sensor detects tensile force acting on the guidewire or a deformation of the extension arm. In an alternative embodiment, the leak is identified using a flow sensor provided at the distal dip of the extension arm which recognizes the PVL jet. In one embodiment, in case of a steerable extension arm, the steering mechanism can be actuated by an automated mechanism to place the distal opening of the extension arm in the position where the strongest flow occurs, e.g. in the center of a PVL jet.

In one embodiment, the system may include a detector module other than a floater combined with a force sensor configured to identify the position of a leak within the heart, the vasculature and/or within a transition area between the heart or the vasculature and the implant, wherein the detector module may include, for example, an optical sensor, a force sensor, pressure sensor and/or a flow sensor

In one embodiment of the method, the extension arm is manually or automatically rotated during looking for a position of a leak within the heart, the vasculature and/or within the transition area between the heart or the vasculature and the implant.

In one embodiment, the occluding member is positioned within the leak at the identified position thereby closing the leak. The occluding member can be advanced to the position of the leak over a guidewire using an occluder catheter within the guiding sheath and the extension arm.

In the last step, the catheter system is removed from the heart and/or vasculature of the patient.

In one embodiment, the above method can be automated using an electronic control unit (ECU) connected to the catheter system having a processor. The ECU includes algorithms that realize the method steps explained above and below. The automatic execution includes, for example, a watch hand type movement of the pre-defined bent or steered extension arm along a circumference of a circle. This step can be actuated by a mechanism rotating the extension arm at a certain continuous speed or in incremental steps. The watch hand type incremental movement can be triggered by a pre-defined signal of the heart's cycle of the patient, for example, such that a) the distal tip of the extension arm is moved (e.g. rotated) while the valve is closed or b) the distal tip of the extension arm is moved (e.g. rotated) while the valve is open. Embodiment a) allows to move the extension arm (and, if applicable, a floater) into a PVL jet and to detect in which angular position the PVL jet is present and how strong it is. Embodiment b) allows for detecting whether there is a jet at a specific angular position. However, movement at a continuous speed can bear the risk to miss a leak when the leak is passed while there is no flow through the leak because of the phase of the heart cycle at this point in time. In one embodiment, the extension arm movement can be triggered at every cycle or after a pre-defined number of cycles left between each movement. In an alternative embodiment, the movement may also be performed independently of the heart's cycle of the patient. In one embodiment, rotation step increments can be in the range from 1° to 90°, preferably in the range from 5° to 60°, more preferably from 15° to 45°.

In one embodiment, the catheter system may automatically place the distal tip of the extension arm adjacent to the upstream opening of a leak (PVL) and align it with the flow axis of the leak by using the position data identified by the catheter system to locate the leak. Further an automated steering system integrated and/or connected with the catheter system can be used for placing the distal tip of the extension arm at this position. Once the position is taken, the guidewire carrying the occluding member can be advanced into and optionally through the leak (automated or by hand) or the occluding member delivery catheter can be advanced over the guidewire for placement of an occluding member.

In one embodiment, at least one radiopaque marker can be attached to various components of the system to facilitate the method by improving the visibility and indicating critical locations such as the tip of the extension arm, the axial location of the alignment component, the periphery of the alignment component, the occluding member, the floater, etc.

can be

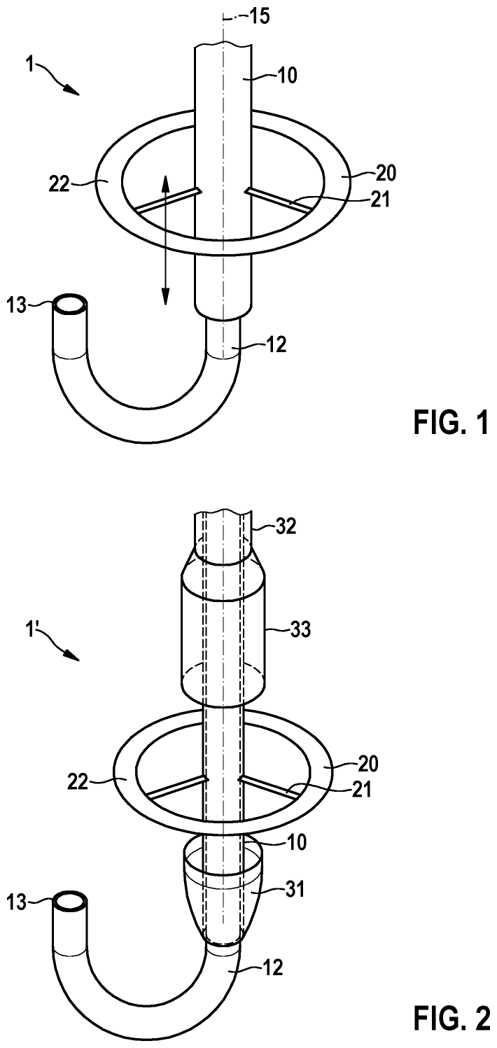

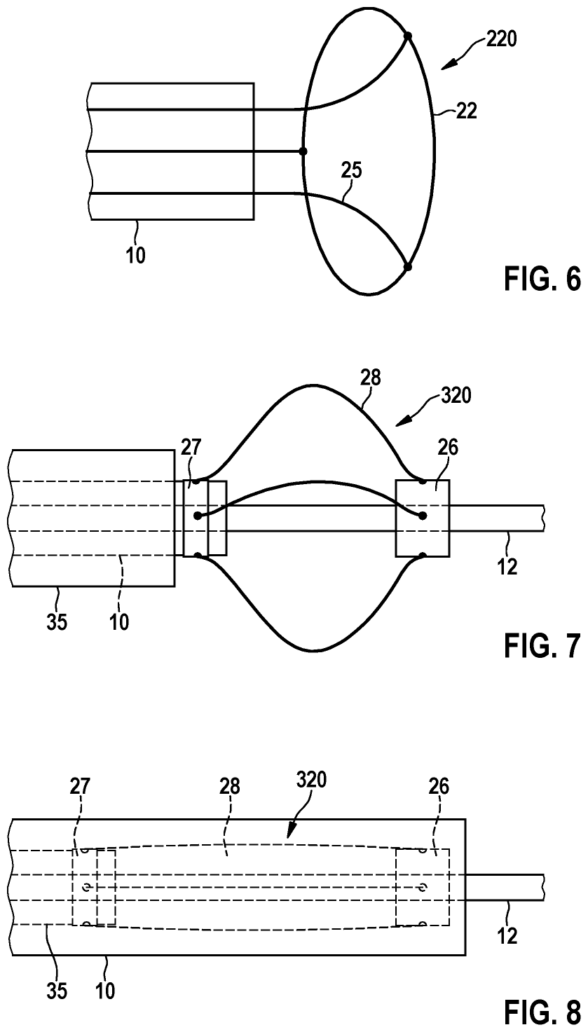

FIG. 1 illustrates a distal portion of a catheter system 1 in accordance with a first embodiment. The catheter 1 has an elongated tubular guiding sheath 10 and a steerable and/or bendable tubular extension arm 12, which may connect with a handle including a steering mechanism at a proximal end (not illustrated). As a result, the catheter may control deflections of the depicted distal tip 13 of the extension arm 12, for example by 180°. This can be for example realized by building a catheter that has a shaft (e.g. made of metal) with a special cutting pattern that allows more bending in a certain direction (embedded or coated with polymer) and by using a pull wire that is attached distally and extends proximally up to a control element. By tightening the pull wire, the catheter then curves. The extension arm 12 is further movable in longitudinal direction and rotatable with regard to the guiding sheath 10. Furthermore, at a distal section of the guiding sheath 10 the catheter system 1 includes an alignment component 20 in an expanded state having three radially extending struts 21 attached to the guiding sheath 10 and one strut 22 running circular around a longitudinal axis 15 of the extension arm 12 and of the guiding sheath 10. The circular strut 22 is fixed to the outer tip of each radial strut 21. The alignment component 20 is located at a distance d from the distal end of the guiding sheath 10 (see FIG. 1). One possibility to attach the alignment component 20 to the guiding sheath is on continuous wires which would run between shafts 10 and 12 and exit through openings in shaft 10 (as indicated in FIG. 6) or by welding or gluing it to the shaft 10, for example.

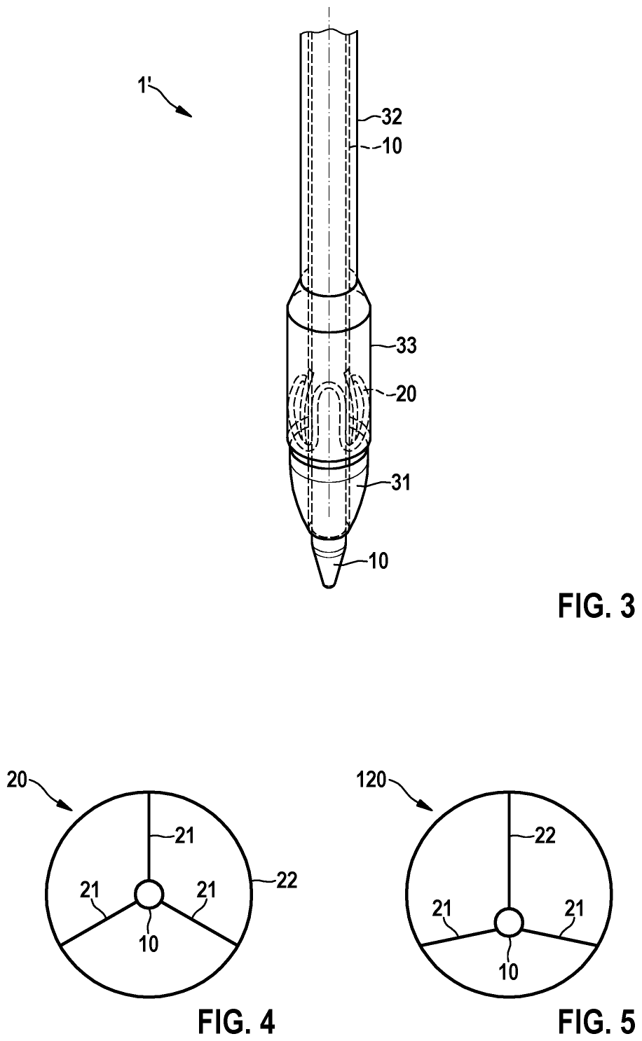

A second embodiment of a catheter system 1′ shown in FIGS. 2 and 3 corresponds to the embodiment of FIG. 1 but additionally includes an outer sheath having a distal part 31 and a proximal part 32, wherein the proximal part 32 forms a chamber 33 having a greater inner and outer diameter than a proximal section of the proximal part 32 of the outer sheath for receipt of the collapsed alignment component 20. The distal part 31 of the outer sheath is formed such that it realizes an atraumatic tip. The atraumatic tip of the distal part 31 functions as an insertion aid and can be collapsed and removed after the distal end of the catheter system is advanced to the targeted area. FIG. 2 shows the expanded state of the alignment component 20 which is released from the outer sheath by retracting the proximal part 32 from the distal part 31. FIG. 3 depicts the collapsed state of the alignment component 20 in which the chamber 33 formed by the outer sheath covers the collapsed alignment component 20 which is folded. The folding can be similar to TAVI when a prosthesis is loaded or recaptured after partial release. When a self-expanding structure (e.g. made of shape memory material, e.g. nitinol) is captured into a capsule, the structure is compressed. Depending on the cutting pattern and the type of trapping of the structure, a compressed structure arrangement is formed. Additionally, the extension arm is retracted within the guiding sheath 10.

Two embodiments of an alignment component 20, 120 are compared in FIGS. 4 and 5. FIG. 4 shows the alignment component 20 of the embodiments of the catheter system of FIGS. 1 to 3, wherein the alignment component 20 includes three radial struts 21 of equal length thereby centering the guiding sheath 10 in the center of the circular strut 22. In contrast, in the embodiment of an alignment component 120 shown in FIG. 5 the guiding sheath 10 is located out of center of the circular strut 22 realized by radial struts 21 of different length. Such construction can be advantageous if the anatomical situation of the patient does not allow centered position of the guiding sheath 10.

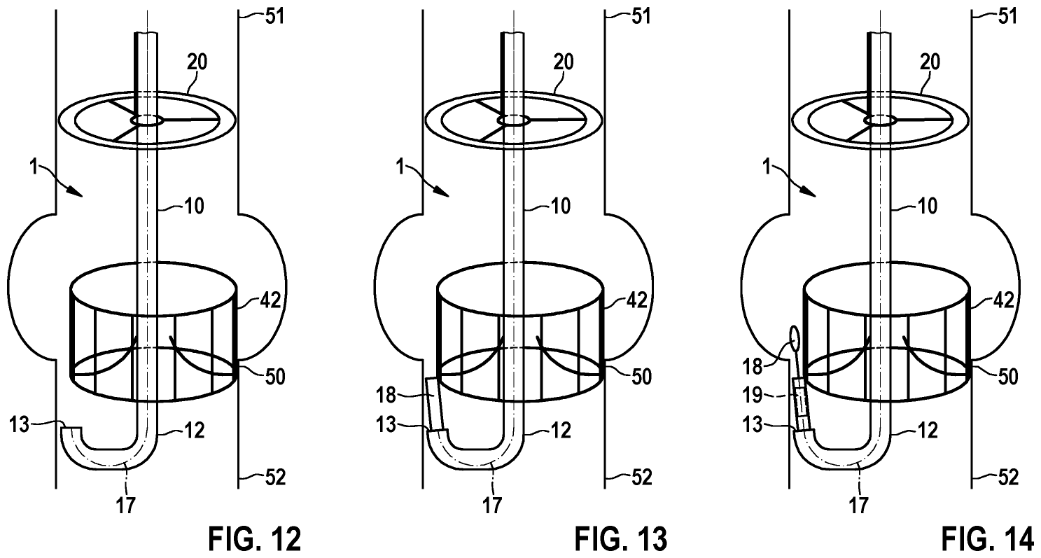

FIG. 11 shows two positions at which an alignment component 20 as shown in FIG. 1, for example, can be placed with regard to an implant in form of a heart valve prosthesis, for example implanted by a transcatheter aortic valve implantation (TAVI). For greater clarity, in FIG. 11 the stent-like structure 40 supporting the artificial heart valve is shown, only. In FIG. 11 by a dashed line two possible positions of the alignment component 20 are indicated within the inflow section and the outflow section of the heart valve prosthesis where it does not hinder the function of the leaflets of the heart valve prosthesis. Alternatively, the alignment component is positioned outside the implant and directly supported by the heart or vasculature of the patient as shown in FIGS. 12 to 14 (see below).

Another alignment component 220 is shown in FIG. 6 having a similar circular strut 22 compared with the embodiments of FIGS. 4 and 5. Further, the circular strut 22 is attached to curved struts 25 attached to the inner surface of the guiding sheath 10. Each curved strut 25 includes an inclined section at its distal end running inclined to the longitudinal axis 15 of the extension arm 12 and the guiding sheath 10.

Another embodiment of an alignment component 320 is depicted in FIGS. 7 and 8. In this embodiment, the alignment component 320 includes a distal sleeve 26 and a proximal sleeve 27, wherein the proximal sleeve 27 is fixed at the guiding sheath 10 and the distal sleeve 26 is movable in longitudinal direction with regard to the proximal sleeve 27. The distal sleeve 26 and the proximal sleeve 27 are connected by three struts 28. FIG. 8 shows the collapsed state of the alignment component 320 in which the three struts 28 run parallel to the longitudinal axis 15 (see FIG. 1) and where the alignment component is covered by a tubular, single piece outer sheath 35. FIG. 7 shows the expanded state in which the outer sheath 35 is retracted and the three struts 28 are bent so that they together realize a bulbous form.

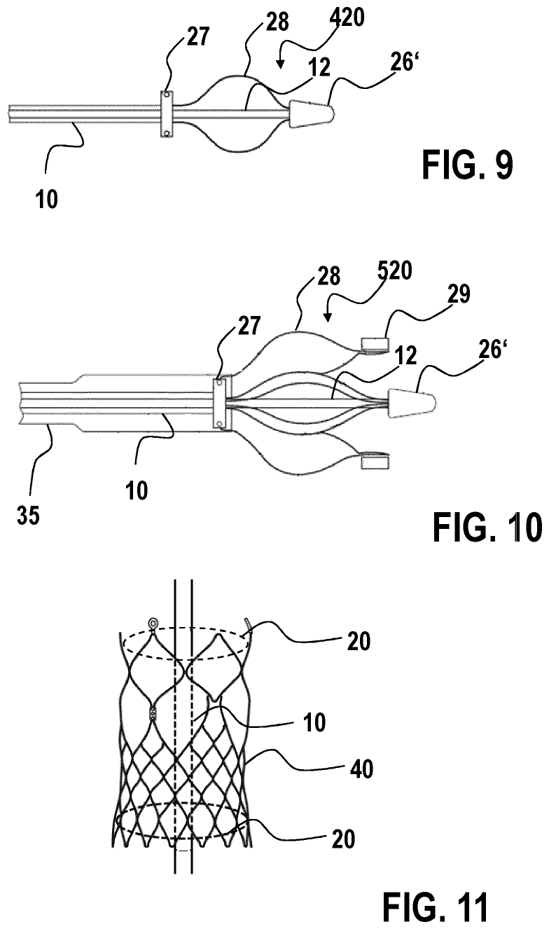

Another embodiment of an alignment component 420 is shown in FIG. 9 which is quite similar to the one of FIGS. 7 and 8. This embodiment includes a distal tip 26′ instead of the distal sleeve 26 to which the struts 28 are attached. FIG. 9 shows the expanded state.

Another similar embodiment of an alignment component 520 is shown in FIG. 10 in the expanded state. A part of the struts 28 is attached to a distal tip 26′ as in FIG. 9. Another part, for example two struts 28, is attached to one distal pad 29 which is accommodated with a pre-defined radial distance to the centered distal tip 26′, wherein the shown alignment component 520 includes two of these pads 29. The alignment component 520 includes at least two of such pads 29. Each pad 29 is supported at the inner wall of the heart, vasculature or implant. The pad 29 is a cylindrical or cuboid element including the material.

FIGS. 12 to 14 show steps of an embodiment of a method for the closure of a PVL at a transition area between the natural heart valve structure 50 and the implant forming a heart valve prosthesis, more particularly a prosthetic aortic valve 42.

At first, a guidewire preferably having a soft tip is advanced through a vascular access site, the aorta 51 and the prosthetic aortic valve 42 into the left ventricle 52. Then, the catheter system 1 as shown in FIG. 1 is advanced over the guidewire until the distal tip of the guiding sheath 10 is placed in the area of the prosthetic aortic valve 42. Afterwards, the alignment component 20 of the catheter system is deployed and/or in the ascending aorta 51 as shown in FIGS. 12 to 14. By the alignment component 20, the distal end of the catheter system has a well-defined, centered position with regard to the prosthetic aortic valve 42 thereby providing a higher reliability to the method for the closure of the PVL and allowing the prosthetic aortic valve 42 to work properly during the surgery. Alternatively, the alignment component can be deployed in the inflow tract of the prosthetic aortic valve 42. In the next step, as shown in FIG. 12, the extension arm 12 is deployed reaching out from the center of the prosthetic aortic valve 42 to the periphery of the valve and forming a bend of about 180° to allow for identifying and positioning an occluding member from the ventricular side of the prosthetic aortic valve 42. The extension arm can be rotatable and/or steerable, alternatively it can be pre-shaped. In the next step, which is shown in FIG. 12 a floater 18 is deployed from the extension arm 12, wherein the floater 18 is attached to a floater guidewire 17. The guidewire 17 and/or the extension arm 12 is/are connected to a force sensor (not shown) located at the proximal end of the catheter system. By rotation of the extension arm around its longitudinal axis 15 it is detected whether a PVL jet flow force deforms the extension arm 12 and/or acts on the guidewire 17 thereby identifying a leak. This indicates that the extension arm 12 is moved along the periphery of the prosthetic aortic valve 42 until the floater 18 is caught by the bloodstream and carried through the PVL which is detected by the force sensor. The position of the extension arm 12 at which the force sensor provides a deformation signal is kept for the following steps of the catheter system 1. In the next step, an occluder delivery catheter having an occluding member 19 at its distal end is advanced through the guiding sheath 10 and the extension arm 12 along the floater guidewire 17 and finally through the PVL (see FIG. 14). Now, the occluding member 19 is deployed in the PVL and the floater 18 and floater guidewire 17 are removed from the vascular system. Alternatively, the floater 18 and a distal section of the floater guidewire 17 are detached from the proximal section of the floater guidewire 17 and left at the PVL site and attached to the occluding member 19. Finally, after the whole circumference of the prosthetic aortic valve 42 is investigated regarding a possible PVL, the delivery components, i.e. the catheter system 1, are removed from the vasculature of the patient, as well.

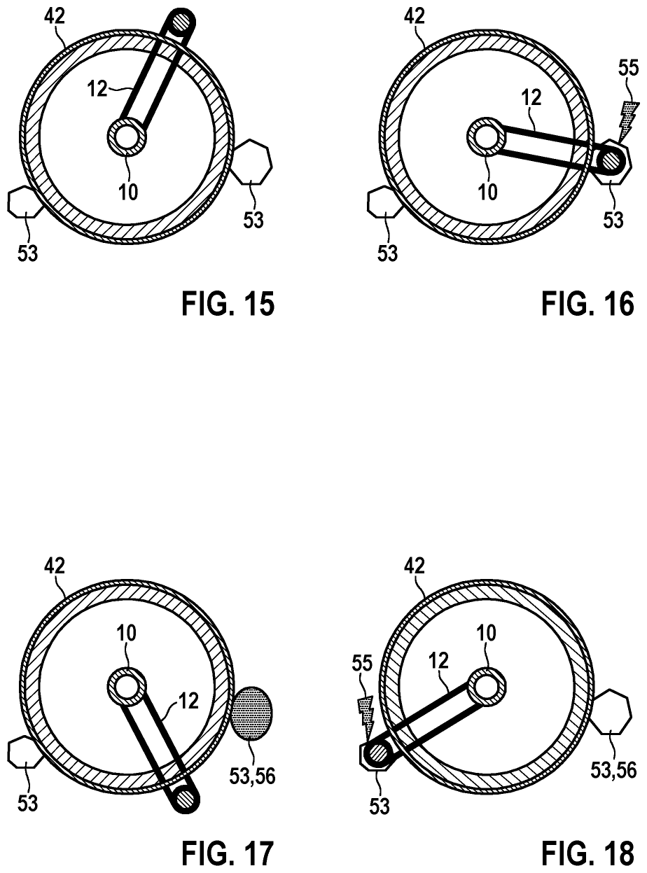

With regard to FIGS. 15 to 18 another embodiment of method for the closure of a PVL is explained. At first, guidewire, preferably having an atraumatic tip is advanced through a vascular access site, the aorta and the prosthetic aortic valve into the left ventricle. Then, the catheter system 1 including a central guiding sheath 10 is advanced over the guidewire until the distal tip is placed in the region of the prosthetic aortic valve. In one embodiment the catheter system 1 has a guidewire lumen separate from the guiding sheath 10 lumen and the guidewire lumen has a distal exit port which is located in a position where the guidewire exits along the valvular flow axis into the left ventricle also when the extension arm 12 is deployed. In another embodiment the guidewire lumen is arranged in the center of the catheter system and the guidewire used for placement of the catheter system can further be used as guidewire for placement of the occluding member after deployment of the extension arm 12. In the next step, the alignment component 20 is deployed in the inflow tract of the prosthetic aortic valve 42 and/or in the ascending aorta. In the next step, an extension arm 12 is deployed reaching out from the center of the prosthetic aortic valve 42 to the periphery of the valve and forming a bend of about 180° to allow for positioning an occluding member 56 from the ventricular side of the prosthetic aortic valve 42. Then, the extension arm is moved along the periphery of the prosthetic aortic valve 42 by rotation of the extension arm 12 and/or by manipulating a steering mechanism as shown in FIGS. 15 to 18. The extension arm 12 includes a PVL jet detector in the region of the exit port at the distal tip of the extension arm 12 which detects the flow and/or force of a jet of a PVL on the catheter during diastole and provides a signal to show the user when the exit port of the catheter at the tip of the extension arm 12 is located close to a leak 53 which is symbolized by arrow 55 in FIG. 16 or 18. In an alternative embodiment, in case the extension arm 12 is steerable, the steering mechanism can be actuated by an automated mechanism to place the distal opening of the extension arm 12 in the position where the strongest flow occurs, e.g. in the center of a PVL jet. When the extension arm 12 is positioned at the location of a leak 53, an occluder guidewire that may include an atraumatic tip is advanced from the ventricular side through the leak into the aortic root and optionally further into the ascending aorta. Now an occluder delivery catheter is advanced within the guiding sheath and the extension arm along the occlude guidewire through the leak 53. An occluding member 56 is deployed in the leak 53 thereby closing the leak 53 (see FIG. 17). Then, the extension arm 12 is further rotated along the periphery of the prosthetic aortic valve 42 further looking for a second leak 53 (see FIG. 18). After examination of the full periphery of the prosthetic aortic valve 42 and closing of all detected leaks the occlude guidewire, the occluder delivery catheter, catheter system and the guidewire are removed from the vasculature of the patient.

As shown by the above embodiments, the catheter system allows for precise and efficient location of a PVL and deployment of an occluding member in the PVL to seal the leak.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A catheter system for an interventional procedure, comprising:
a bendable and/or steerable extension arm;
a guiding sheath comprising a distal section, wherein the extension arm is fixed at a distal end of the distal section of the guiding sheath or is configured to be moved along the guiding sheath so that it projects in an operation position from the distal end of the distal section;
an inner lumen in the extension arm through which a guidewire can be maneuvered, wherein the inner lumen defines a longitudinal axis;
at least one alignment component having a collapsed state and being configured to expand into an expanded state, wherein the alignment component is attached to the guiding sheath at a pre-defined position within the distal section but proximally from its distal end or the alignment component is configured to be advanced along the guiding sheath to this pre-defined position within the distal section;
at least one guidewire within the inner lumen of the extension arm; and
an occluding member in contact with a distal guidewire section and/or a distal guidewire end of the at least one guidewire.

2. The catheter system of claim 1, wherein the alignment component is configured to position the distal end of the guiding sheath into a pre-defined position.

3. The catheter system of claim 1, comprising a detector module configured to identify the position of a leak within a heart, a vasculature and/or within a transition area between the heart or the vasculature and an implant.

4. The catheter system of claim 1, wherein the alignment component comprises one or more of a self-expanding structure and a mechanically expandable structure.

5. The catheter system of claim 1, wherein the occluding member comprises a coil.

6. The catheter system of claim 1, wherein the alignment component comprises an outer diameter that is in a range between 20 mm and 50 mm in the expanded state.

7. The catheter system of claim 1, wherein the extension arm is configured to be manually or automatically rotatable and/or steerable around its longitudinal axis relative to the guiding sheath.

8. The catheter system of claim 1, wherein the guiding sheath is bendable by at least 120°.

9. The catheter system of claim 1, wherein the alignment component comprises a plurality of struts distributed on the circumference, wherein each strut of the plurality of struts runs at least sectionwise into radial direction and/or inclined with regard to the longitudinal axis in the expanded state thereby producing a force directed radially outward.

10. The catheter system of claim 1, wherein the alignment component comprises at least a section of a circular strut and spoke-like struts.

11. The catheter system of claim 1, comprising a floater attached to a distal guidewire end of the at least one guidewire.

12. A method for the closure of at least one paravalvular leakage within a targeted area of an heart, a vasculature and/or a transition area between the heart or the vasculature and an implant fixed at the patient's heart or vasculature, the method comprising the following consecutive steps:
advancing a guidewire through a vascular access site, an aorta or a prosthetic aortic valve into the left ventricle of a patient's heart,
advancing a distal section of a guiding sheath of a catheter system, over the guidewire to a targeted area, wherein an alignment component of the catheter system is in a collapsed state,
expanding the alignment component at a pre-defined position within the distal section of the guiding sheath and with regard to the implant and/or the heart or vasculature such that it is supported at the implant, heart and/or vasculature,
using a bendable and/or steerable extension arm fixed at or projecting from the distal end of the distal section of the guiding sheath to identify a position of a leak within the heart, the vasculature and/or within the transition area between the heart or the vasculature and the implant, and
advancing an occluding member through an inner lumen of the extension arm to the leak at the identified position using a guidewire and occluding the leak.

13. The method of claim 12, comprising identifying the position of the leak is identified by using a floater being attached to a distal end of the guidewire.

14. The method of claim 12, comprising rotating the extension arm within the heart, the vasculature and/or within the transition area between the heart or the vasculature and the implant, when looking for the position of a leak.

15. The method of claim 12, comprising monitoring wherein the tensile force acting on the guidewire and/or the extension arm.

16. The catheter system of claim 2, wherein the pre-defined position comprises one of an approximately central position or a position having a pre-set distance from the central position, with regard to a surrounding heart, vasculature or implant.

17. The catheter system of claim 3, wherein the detector module comprises one or more of optical sensor, a force sensor, pressure sensor and a flow sensor.
